(19) United States
(12) Patent Application Publication
(10) Pub. No.: US 2026/0102491 A1
(43) Pub. Date: Apr. 16, 2026

(54) METHOD FOR IN VITRO LARGE-SCALE EXPANSION OF DOUBLE-NEGATIVE T CELLS RICH IN TSCM AND TCM

(71) Applicant: Ruichuang Biotech. Co., Ltd., Shaoxing, Zhejiang (CN)

(72) Inventors: Liming YANG, Shaoxing, Zhejiang (CN); Dan WANG, Shaoxing, Zhejiang (CN); Liuyang WANG, Shaoxing, Zhejiang (CN); Xiancai LI, Shaoxing, Zhejiang (CN); Jianjun TONG, Shaoxing, Zhejiang (CN)

(21) Appl. No.: 18/707,447

(22) PCT Filed: Nov. 4, 2022
(86) PCT No.: PCT/CN2022/130054

§ 371 Date: May 3, 2024

(30) Foreign Application Priority Data
Nov. 5, 2021 (CN) 202111308473.4

(51) Int. Cl. A61K 40/11; A61K 40/50; C12N 5/00; C12N 5/0783

(52) U.S. Cl. CPC A61K 40/11; A61K 40/50; C12N 5/0087; C12N 5/0636; C12N 2500/24; C12N 2500/90; C12N 2501/2302; C12N 2501/2305; C12N 2501/2321; C12N 2501/33; C12N 2501/515; C12N 2501/999

(57) ABSTRACT

The present invention relates to a method for the in vitro large-scale expansion of double-negative T cells rich in Tscm and Tem. Specifically, the method comprises the step of: (a) providing an initial sample I of peripheral blood obtained from a donor, (b) pretreating the initial sample I to obtain sample II, and (c) culturing sample II in a culture system containing a culture medium suitable for the growth of DNT cell, thereby obtaining a sample III. The expansion process of the present invention is simple and stable, and can achieve the in vitro large-scale culturing of DNT cells rich in Tscm and Tcm cell characteristics. The purity of the final product DNT cells (CD3+CD4−CD8−) is ≥85%, which meets the requirements for clinical use.

### TECHNICAL FIELD

The present invention relates to the field of cell therapy technology, specifically relates to a method for in vitro large-scale expansion of double-negative T cells rich in Tscm and Tcm.

### BACKGROUND

Adoptive immune cell therapy is an emerging tumor treatment with significant efficacy, and is a new type of treatment for immune anti-cancer. It is the use of biotechnology and biological agents to collect immune cells from patients or healthy donors for activation and amplification in vitro, and then infuse back into the patient's body to stimulate and enhance the patient's autoimmune function, so as to achieve the purpose of treating tumors.

According to the literature and clinical data, the key factors determining the durability and anti-tumor effect of adoptive cell immunotherapy are closely related to the cellular memory differentiation state before infusion, that is, the proportion of Tscm cells and Tcm (Central Memory T) cells in the cell product. According to the latest research of memory T cell differentiation pattern, native T cells are stimulated by antigens to first differentiate into stem cell-like memory T cells (Tscm), then differentiate into central memory T cells (Tcm), and finally into Effector Memory T cells. Tscm cells and Tcm cells have strong stem cell-like characteristics, which make them maintain a certain degree of self-renewal, differentiation and long-term survival, and have strong IFN-γ secretion ability, which can exist in the body for a long time and play a long-term anti-tumor role.

Double negative T (DNT) cells refer to the CD3+CD4−CD8−mature T lymphocyte subset that normally exists in peripheral blood, accounting for about 1-3% of peripheral blood mononuclear cells. DNT cells express CD3 molecules and αβ- or γδ-T cell receptor (TCR) on the surface, but do not express CD4 and CD8 molecules, and do not respond to constant invariant Nature Killer T (INKT) cell-specific αGalCer, so they are different from conventional T cells, NK cells, and NKT cells. DNT cells exert cytotoxicity and antigen presentation functions through a variety of natural human mechanisms, while releasing cytokines/chemokines to activate a broader immune response. It has been reported that DNT cells have cytotoxic activity for a variety of tumors, and have good in vitro and in vivo tumor killing effects in AML, lymphoma, cervical cancer, lung cancer, liver cancer, gastric cancer and other blood or solid tumors, and DNT cells also play an important role in immune regulation, immunosuppression and autoimmune diseases. DNT cells kill tumor cells without being restricted by the Major Histocompatibility Complex (MHC) molecule, and the allogeneic DNT cells derived from healthy donors expanded in vitro have no killing toxicity to normal cells, do not affect the further differentiation of hematopoietic stem cells, and do not cause graft-versus-host disease (GvHD), and have no response for host versus graft (HvG), which makes it a promising candidate for the clinical treatment of tumors.

In the first place, the expansion of DNT cells was based on the small-scale expansion method used in laboratory research, and its clinical application was limited by the small culture scale and the addition of animal-derived serum, and the subsequent in vitro expansion system of cells was developed to remove the animal-derived product, but still needed to add autologous plasma and/or AB serum and/or human blood albumin, and soluble anti-human CD3 monoclonal antibody was added as an activator throughout the culture cycle. The latter is currently commercially available for GMP-grade products of mouse origin (clone number: OKT3), which brings potential safety risks to the clinical application of the product. In the past, the proportion of Tscm cells and Tcm cells in DNT cells expanded in vitro was also very low, and it was not mentioned in previous literature and patents.

Therefore, there is a need for a method for large-scale expansion of double-negative T cells rich in Tscm and Tcm cell characteristics in vitro.

### SUMMARY OF THE INVENTION

The purpose of the present invention is to provide a method for large-scale expansion of double-negative T cells rich in Tscm and Tcm cell characteristics in vitro.

The first aspect of the present invention provides an expansion method for double-negative T cells enriched with Tscm cells and Tcm in vitro, comprising steps:
(a) providing an initial sample I of peripheral blood obtained from a donor;
(b) pretreating the initial sample I to obtain sample II;
(c) culturing sample II in a culture system containing a culture medium suitable for the growth of DNT cell, thereby obtaining a sample III, wherein one or more cytokines selected from the following group are added to the culture system:

5-50 ng/ml recombinant human interleukin 21, 1-10 ng/ml recombinant human interleukin 1β, 5-50 ng/ml recombinant human interleukin 7, 5-50 ng/ml recombinant human interleukin 15, 5-50 ng/ml recombinant human interleukin 12;
wherein, in step (c), anti-human CD3 antibody is not added to the culture system.

In another preferred embodiment, step (c) also includes the following steps:
(d) in a culture system containing medium suitable for DNT cell growth, sample III is cultured to obtain the required amount of Tscm and Tcm-rich DNT cells, as sample IV; wherein, one or more cytokines selected from the following group are added to the culture system:
5-50 ng/ml recombinant human interleukin 7, 5-50 ng/ml recombinant human interleukin 12 or 5-50 ng/ml recombinant human interleukin 15; and
(e) collecting sample IV from a solution suitable for DNT cell preservation.

In another preferred embodiment, the pretreatment in step (b) comprises:
(b1) CD4+ and CD8+ T cells are removed from the starting sample I, resulting a starting sample I with CD4+ and CD8+ depleted;
(b2) sample II was obtained through activating starting sample I with CD4+ and CD8+ depleted by anti-human CD3 monoclonal antibody in a culture system suitable for DNT cell growth.

In another preferred embodiment, in step (b2), the number of activation day is 2-7 days, preferably 3-6 days.

In another preferred embodiment, in step (b2), the anti-human CD3 monoclonal antibody is selected from a human CD3 monoclonal antibody immobilized on a medium, including but not limited to a human CD3 monoclonal antibody coated on culture media, magnetic beads, or degradable beads.

In another preferred embodiment, in step (b2), a human CD3/CD28 monoclonal antibody coated on degradable beads is used to activate the starting sample I with CD4+ and CD8+ depleted, so that obtained sample II.

In another preferred embodiment, the concentration of the anti-human CD3 monoclonal antibody is selected from 20 ng/ml-20 μg/ml.

In another preferred embodiment, in step (b), the number of cells of the CD4+ and CD8+ depleted starting sample I is N0;
in step (c), the number of DNT cells in sample III is N1;
in step (d), the number of DNT cells in sample IV is N2, wherein,
N1/N0r of preferably DNT more preferably DNT c most preferably DNT c
N2/N0ably DNT cells in sample more preferably NT c00, most preferably T c00,s

In another preferred embodiment, in step (b), the density of DNT cells in sample II is selected from 1le⁵-1 in⁷ cells/ml, preferably 5ity⁵-8ell⁶ cells/ml, and more preferably 1nd 6-4efe⁶ cells/ml.

In another preferred embodiment, in step (c), the cytokines are selected from one or more of the following groups:

10-1000 IU recombinant human interleukin 2, 5-50 ng/ml recombinant human interleukin 7, 5-50 ng/ml recombinant human interleukin 12, 5-50 ng/ml recombinant human interleukin 15, 5-50 ng/ml recombinant human interleukin 21, 1-10 ng/ml recombinant human interleukin 1β.

In another preferred embodiment, in step (c), the number of culture days is selected from 5-21 days, preferably 6-17 days, and more preferably 7-14 days.

In another preferred embodiment, in steps (b), (c) and (d), the culture system of the medium suitable for DNT cell growth does not contain anti-human CD3 antibody, such as OKT3.

In another preferred embodiment, the culture system in steps (b), (c) and (d) contains 200-1000 IU/mL (preferably 300-700 IU/mL, more preferably 500 IU/mL) of recombinant human interleukin 2.

In another preferred embodiment, in steps (b), (c) and (d), the culture system of the medium suitable for DNT cell growth does not contain serum.

In another preferred embodiment, in steps (b), (c) and (d), the culture system of the medium suitable for DNT cell growth contains serum substitutes selected from the following groups: ICSR (Immune Cell Serum Replacement), KSR (KnockOut Serum Replacement).

In another preferred embodiment, in steps (b), (c) and (d), the concentration (v/v) of the ICSR is 2%-30%.

In another preferred embodiment, the medium was selected from the following group: AIM-V, X-VIVO-10, X-VIVO-15, Aly505, GT551 medium.

In another preferred embodiment, molecules selected from the following groups are also added to the medium: 1-10 μg/ml recombinant human transferrin, 1-10 μg/ml recombinant human insulin, 10-100 μg/ml ascorbic acid, 1-5 μg/ml ethanolamine, 1-5 μg/ml linoleic acid, 1-5 μg/ml oleic acid.

In another preferred embodiment, in step (c), the medium contains:
(1) 200-1000 IU/mL recombinant human interleukin 2;
(2) 0%-20%, preferably 5%-15% (v/v) ICSR;
(3) one or more cytokines selected from the following group:
10-1000 IU, preferably 50-700 IU recombinant human interleukin 2,
1-50 ng/ml, preferably 5-25 ng/ml recombinant human interleukin 7,
1-50 ng/ml, preferably 5-25 ng/ml recombinant human interleukin 12,
1-50 ng/ml, preferably 5-25 ng/ml recombinant human interleukin 15,
1-50 ng/ml, preferably 5-25 ng/ml recombinant human interleukin 21,
1-10 ng/ml, preferably 1.5-8.5 ng/ml recombinant human interleukin 1ß; and
(4) one or more addition factors selected from the following group:
0.5-15 g/ml, preferably 1-10 μg/ml of recombinant human transferrin,
0.5-15 μg/ml, preferably 1-10 μg/m recombinant human insulin,
10-100 μg/ml, preferably 15-60 μg/ml ascorbic acid,
0.5-10 μg/ml, preferably 1-5 μg/ml ethanolamine,
0.5-10 μg/ml, preferably 1-5 μg/ml linoleic acid,
0.1-5 μg/ml, preferably 1.5-2.5 μg/ml oleic acid.

In the second aspect of the present invention, a DNT cell population is provided, and the DNT cell population is prepared by the method described in the first aspect of the present invention.

In another preferred embodiment, the DNT cell population has one or more characteristics selected from the following group:
(a1) 40%-80% of cells were Tscm cells;
(b1) 10%-40% of cells are Tcm cells; or
(a2) 45%-75% of cells were Tscm cells;
(b2) 5% to 35% of cells are Tcm cells; or
(a3) 30%-60% of cells were Tscm cells;
(b3) 15%-40% of cells are Tcm cells; or
(a4) 20%-40% of cells were Tscm cells;
(b4) 20%-30% of cells are Tcm cells.

In another preferred embodiment, the proportion of Tscm cells and Tcm cells in the DNT cell population ≥cells in the DNT cell p more preferably e DNT cell most preferably e DNT

In another preferred embodiment, the DNT cell viability rate ton on population ion t more preferably ll viabili most preferably ll via

In another preferred embodiment, the purity (%) of DNT cells (CD3+) is ≥80%, preferably 90%, more preferably ≥95%, and most preferably ≥97%.

In another preferred embodiment, the purity (%) of the DNT cells (CD3⁺CD4⁻CD8⁻) was e purity (%) of the DNT more preferably (%) of th most preferably (%) of

In the third aspect of the present invention, a use of DNT cells as described in the second aspect of the present invention is provided for the preparation of a pharmaceutical composition or preparation, and the pharmaceutical composition or preparation is used for:
(a) prevention and/or treatment of tumors;
(b) prevention and/or treatment of infectious diseases;
(c) prevention and/or treatment of autoimmune diseases;
(d) prevention and/or treatment of graft-versus-host disease; and/or
(e) modulation of immune responses.

In another preferred embodiment, the tumor is a tumor allogeneic to the DNT cell.

In another preferred embodiment, the tumor selected from the following groups: hematologic tumors, solid tumors, and a combination thereof.

In another preferred embodiment, the hematologic malignancies selected from the following groups: lymphoma (Hodgkins and non-Hodgkins), acute myeloid leukemia (AML), multiple myeloma (MM), chronic lymphocytic leukemia (CLL), acute lymphoid leukemia (ALL), diffuse large B-cell lymphoma (DLBCL), chronic myeloid leukemia (CML), chronic myelomonocytic leukemia (CMML), myelodysplastic syndrome (MDS), and a combination thereof.

In another preferred embodiment, the solid tumors selected from the following groups: gastric cancer, gastric cancer peritoneal metastasis, liver cancer, leukemia, kidney tumors, lung cancer, small bowel cancer, melanoma, bone cancer, prostate cancer, colorectal cancer, breast cancer, colorectal cancer, colorectal cancer, cervical cancer, ovarian cancer, lymphoma, nasopharyngeal cancer, adrenal tumors, bladder tumors, non-small cell lung cancer (NSCLC), glioma, head and neck cancer, pancreatic cancer, and a combination thereof.

In another preferred embodiment, the autoimmune diseases include diabetes mellitus, rheumatoid arthritis, multiple sclerosis, systemic lupus erythematosus, nausea anemia, hemolytic anemia, autoimmune thrombocytopenia, autoimmune liver disease, ankylosing spondylitis, myasthenia gravis, IgA nephropathy, primary renal ice syndrome, psoriasis, vitiligo.

In the fourth aspect of the present invention, a cell preparation is provided, which comprises a population of DNT cells as described in the second aspect of the present invention.

In another preferred embodiment, the cell preparation comprises a population of DNT cells and a pharmaceutically acceptable carrier.

In the fifth aspect of the present invention, a medium suitable for the growth of DNT cells is provided, and the medium comprises one or more cytokines selected from the following group:
5-50 ng/ml recombinant human interleukin 21, 1-10 ng/ml recombinant human interleukin 1β, 5-50 ng/ml recombinant human interleukin 7, 5-50 ng/ml recombinant human interleukin 15, 5-50 ng/ml recombinant human interleukin 12;
wherein, anti-human CD3 antibody is not added to the medium.

In another preferred embodiment, the medium suitable for the growth of DNT cells contains serum substitutes selected from the following groups: ICSR (Immune Cell Serum Replacement), KSR (KnockOut Serum Replacement).

In another preferred embodiment, the medium contains one or more cytokines selected from the following groups:

10-1000 IU recombinant human interleukin 2, 5-50 ng/ml recombinant human interleukin 7, 5-50 ng/ml recombinant human interleukin 12, 5-50 ng/ml recombinant human interleukin 15, 5-50 ng/ml recombinant human interleukin 21, 1-10 ng/ml recombinant human interleukin 1β.

In another preferred embodiment, the medium was selected from the following group: AIM-V, X-VIVO-10, X-VIVO-15, Aly505, GT551 medium.

In another preferred embodiment, the medium contains:
(1) 200-1000 IU/mL recombinant human interleukin 2;
(2) 0%-20%, preferably 5%-15% (v/v) ICSR;
(3) one or more cytokines selected from the following group:
10-1000 IU, preferably 50-700 IU recombinant human interleukin 2,
1-50 ng/ml, preferably 5-25 ng/ml recombinant human interleukin 7,
1-50 ng/ml, preferably 5-25 ng/ml recombinant human interleukin 12,
1-50 ng/ml, preferably 5-25 ng/ml recombinant human interleukin 15,
1-50 ng/ml, preferably 5-25 ng/ml recombinant human interleukin 21,
1-10 ng/ml, preferably 1.5-8.5 ng/ml recombinant human interleukin 1ß; and
(4) one or more addition factors selected from the following group:
0.5-15 μg/ml, preferably 1-10 μg/ml of recombinant human transferrin,
0.5-15 μg/ml, preferably 1-10 μg/m recombinant human insulin,
10-100 g/ml, preferably 15-60 ng/ml ascorbic acid,
0.5-10 μg/ml, preferably 1-5 μg/ml ethanolamine,
0.5-10 μg/ml, preferably 1-5 g/ml linoleic acid,
0.1-5 μg/ml, preferably 1.5-2.5 μg/ml oleic acid.

The sixth aspect of the present invention provides a method for (a) preventing and/or treating tumors, (b) preventing and/or treating infectious diseases, (c) preventing and/or treating autoimmune diseases, (d) preventing and/or treating graft-versus-host diseases, and/or (e) modulating immune responses, which comprising administering DNT cells as described in the second aspect of the present invention to subjects in need.

It should be understood that, within the scope of the present invention, each technical feature of the present invention described above and in the following (as examples) may be combined with each other to form a new or preferred technical solution, which is not listed here due to space limitations.

### DETAILED DESCRIPTION OF THE INVENTION

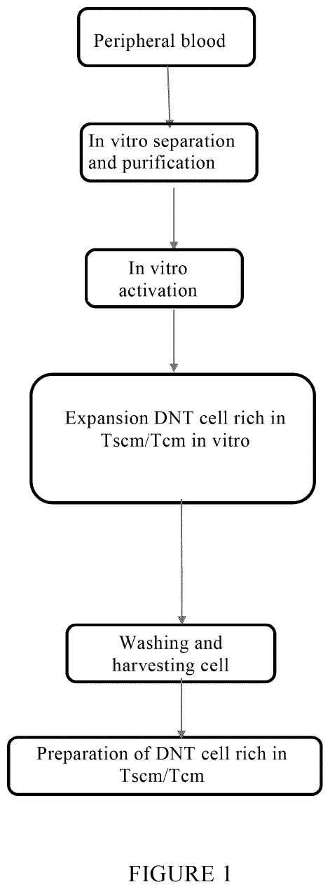

FIG. 1 shows a process flow diagram of the present invention.

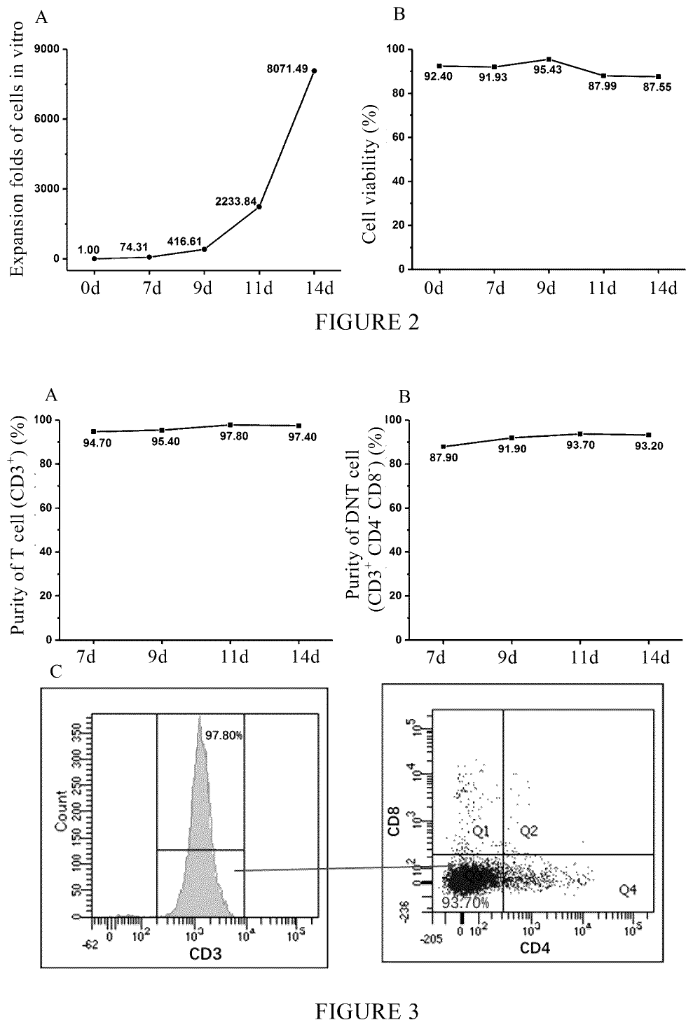

FIG. 2 shows the DNT cell proliferation curve (FIG. 2A) and the cell viability curve (FIG. 2B).

FIGS. 3A and 3B show DNT cell purity curves (CD3%, CD4 CD8%).

FIG. 3C shows the DNT cell purity flow cytometry on day 11.

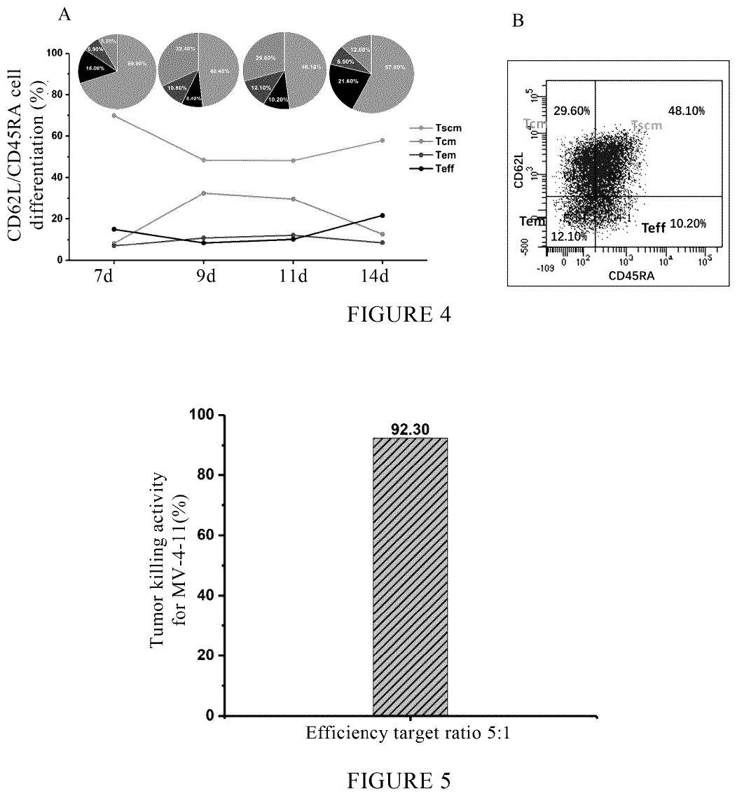

FIG. 4 shows the DNT cell differentiation curve and the flow cytometry of DNT cell differentiation on day 11

FIG. 5 shows the tumoricidal activity of DNT cells at efficiency target ratio of 4:1 to MV411 on day 10 of in vitro expansion for 2 hours co-incubation.

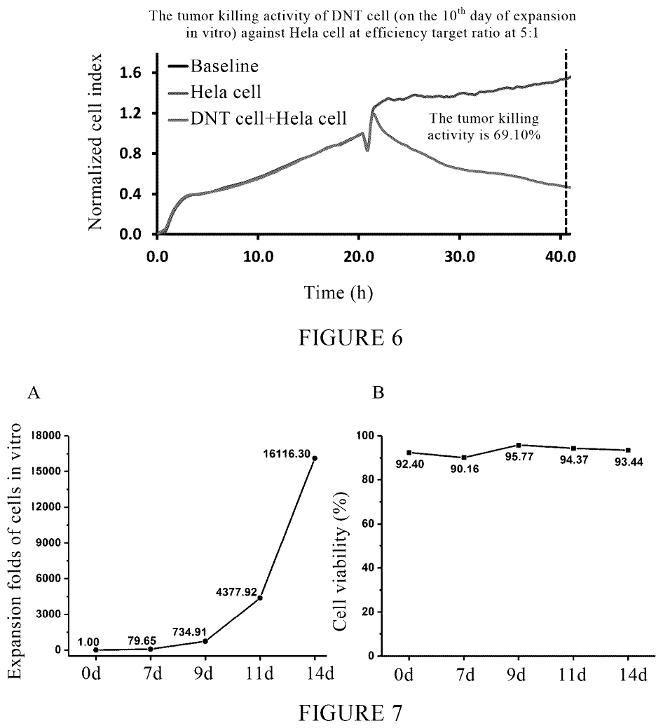

FIG. 6 shows the real-time tumor killing plot of DNT cells at efficiency target ratio of 5:1 with Hela cell efficiency target ratio on day 10 of in vitro expansion co-incubation at 20 hours.

FIG. 7 shows the DNT cell proliferation curve (FIG. 7A) and the cell viability curve (FIG. 7B).

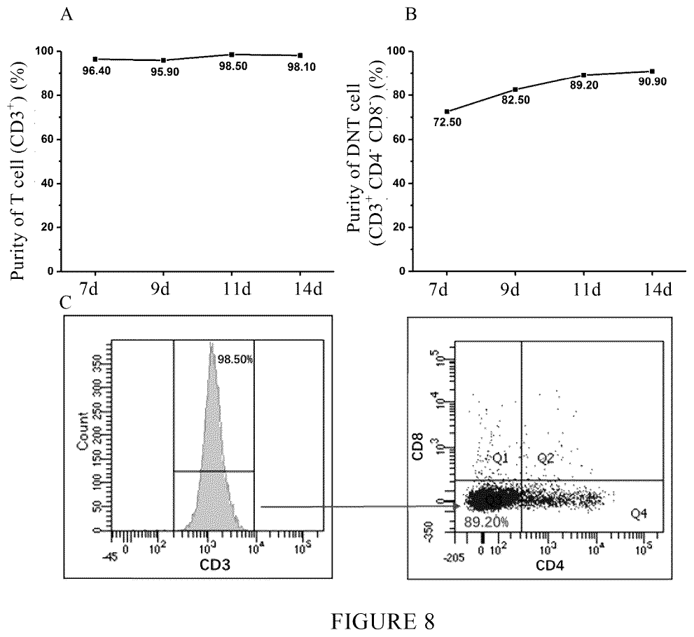

FIGS. 8A and 8B show the DNT cell purity change curve, and FIG. 8C shows the DNT cell purity flow cytometry plot on day 11.

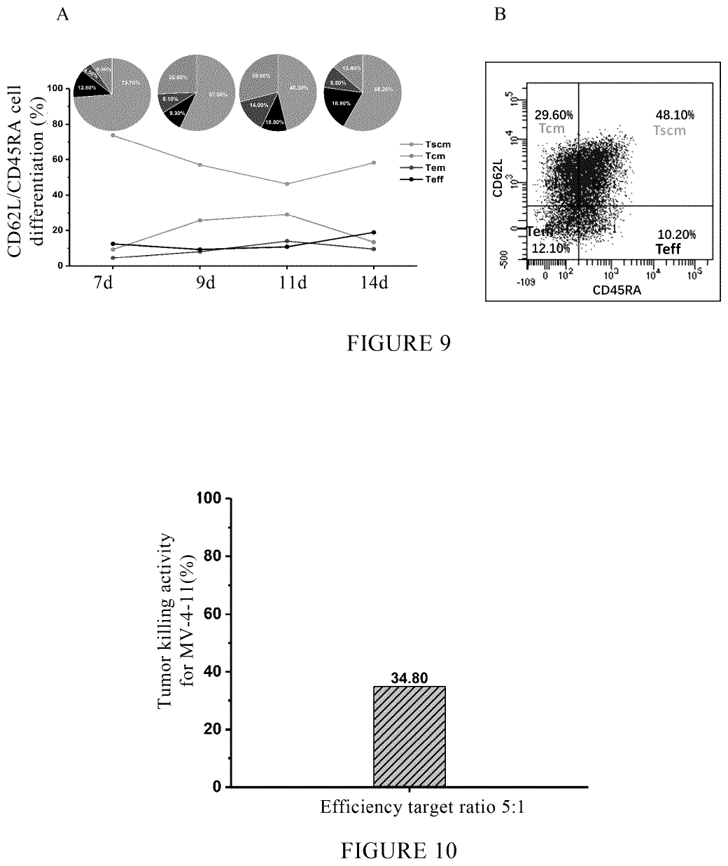

FIG. 9 shows the DNT cell differentiation curve and the flow cytometry of DNT cell differentiation on day 11.

FIG. 10 shows the tumoricidal activity of DNT cells at efficiency target ratio of 4:1 to MV411 on day 10 of in vitro expansion for 2 hours co-incubation.

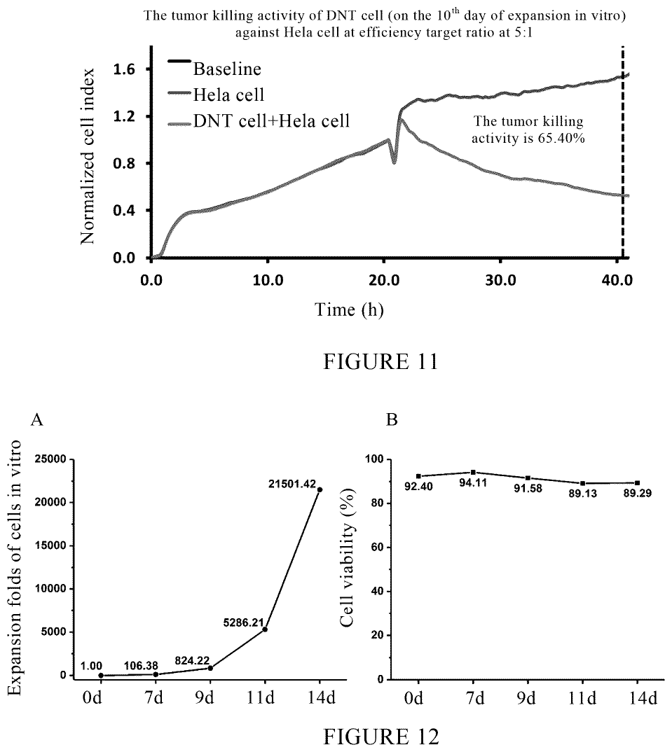

FIG. 11 shows the real-time tumor killing plot of DNT cells at efficiency target ratio of 5:1 to Hela cell on day 10 of in vitro expansion for 20 hours co-incubation.

FIG. 12 shows the DNT cell proliferation curve (FIG. 12A) and the cell viability curve (FIG. 12B).

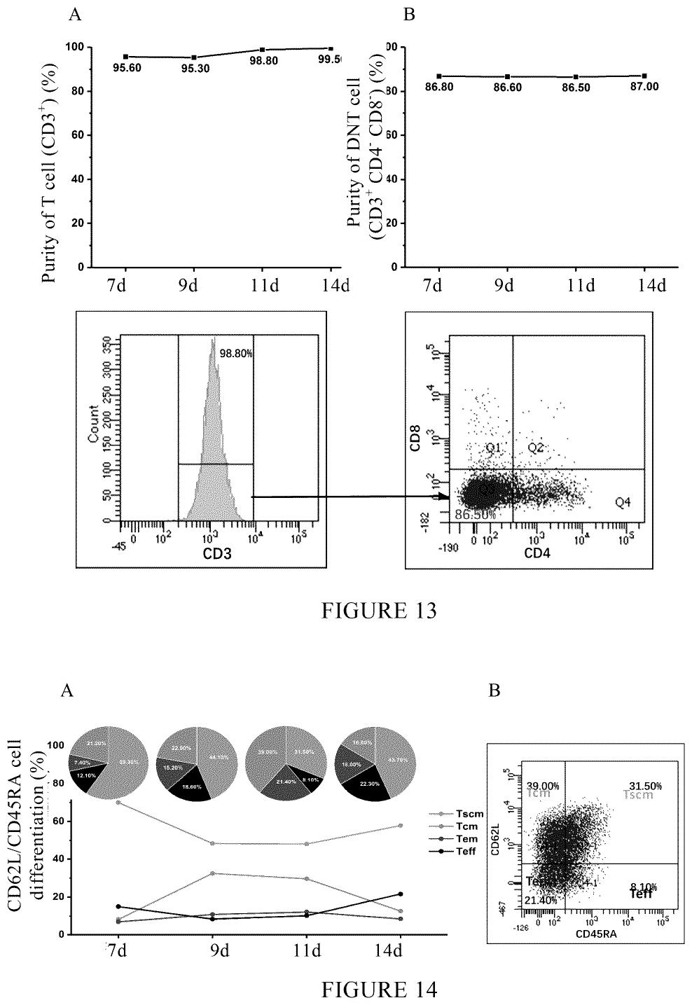

FIG. 13 shows the DNT cell purity curve and the flow cytometry of DNT cell purity on day 11 (FIG. 13C).

FIG. 14 shows the DNT cell differentiation curve and the flow cytometry of DNT cell differentiation on day 11.

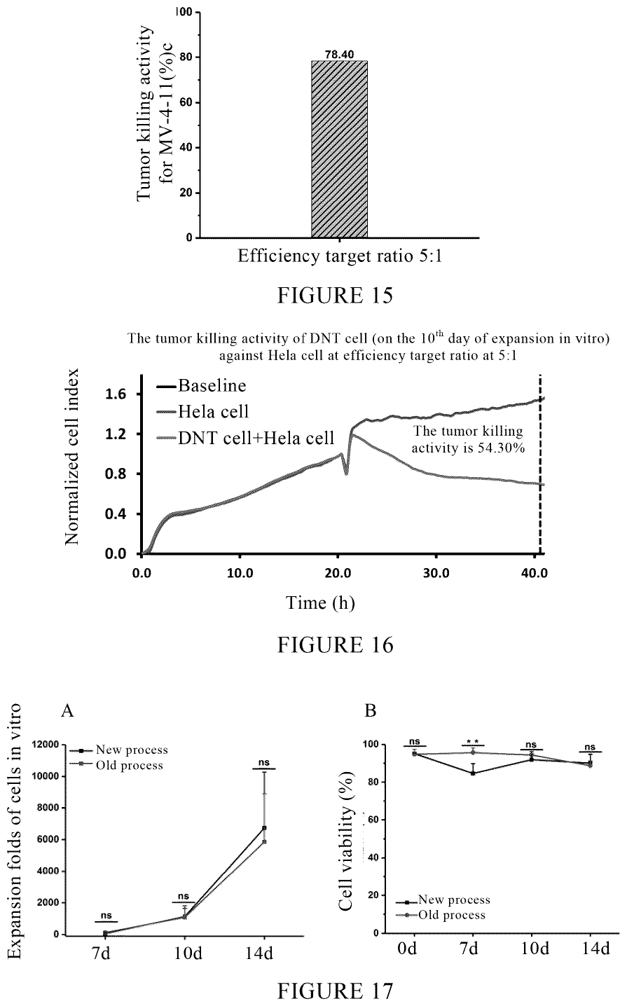

FIG. 15 shows the cell tumoricidal activity of DNT cells at efficiency target ratio of 4:1 to MV411 on day 10 of in vitro expansion for 2 hours co-incubation.

FIG. 16 shows the real-time tumor killing plot of DNT cells at efficiency target ratio of 5:1 to Hela cell on day 10 of in vitro expansion for 20 day co-incubation.

FIG. 17 shows the DNT cell proliferation (FIG. 17A) and cell viability curves (FIG. 17B) (n=4) of the four donors with the new and old processes.

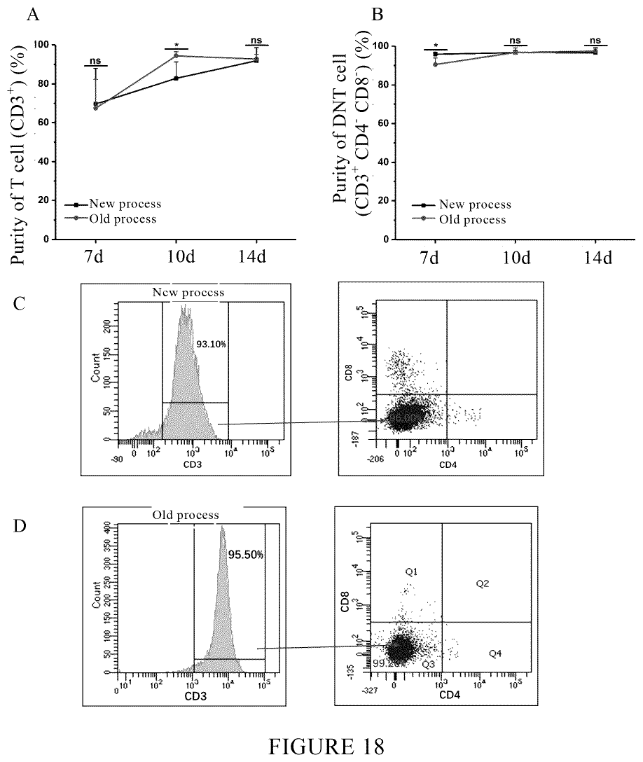

FIG. 18 shows the differential analysis of DNT cell purity curves between the new and old processes (n=4) (18A-B) for the four donors, and the DNT cell purity flow cytometry plots for the new process (18C) and the old process (18D) for donor 2 on day 10.

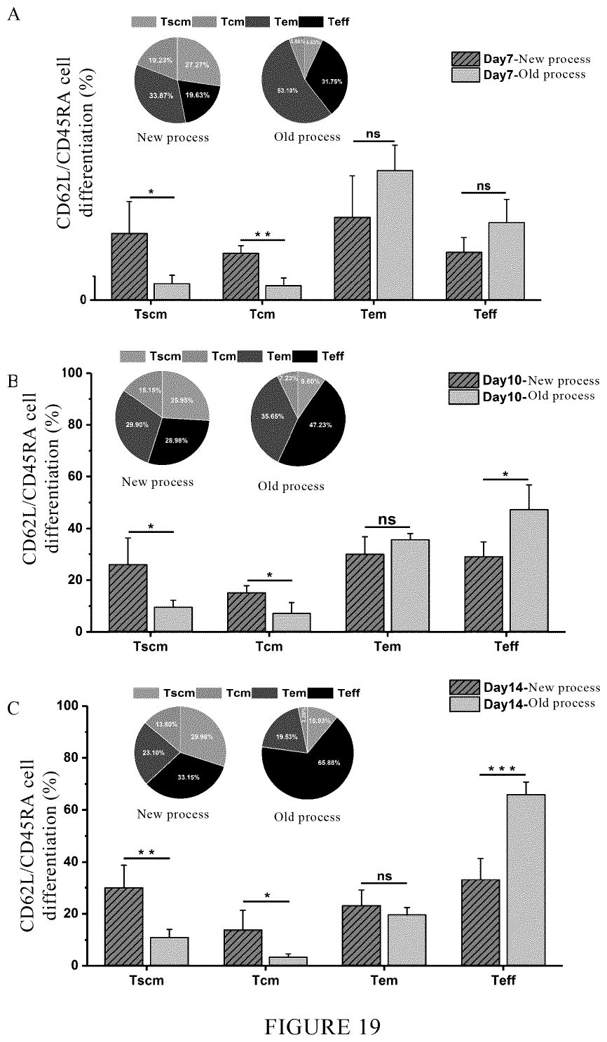

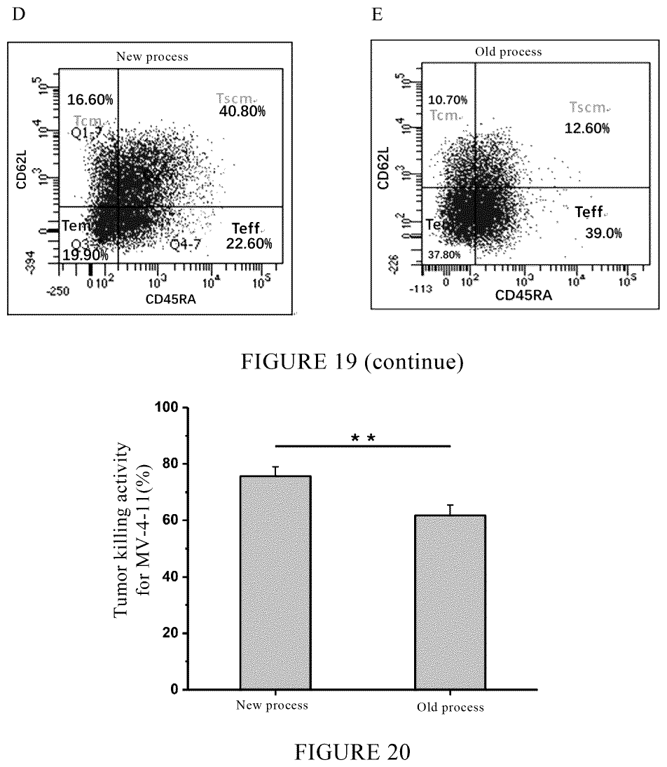

FIG. 19 shows the Tscm/Tcm/Tcm/Toff cell ratio change curves and differential analysis (n=4), fluorescein-labeled anti-human CD45RA/CD62L antibody, which was detected by flow cytometry on the 7th, 10th, and 14th days of DNT cell expansion between the new process and the old process and the flow cytometry of DNT cell Tscm/Tcm/Tcm/Teff cell differentiation from donor 2 on day 10.

FIG. 20 shows the tumoricidal activity of DNT cells at efficiency target ratio of 4:1 to MV411 on day 10 of in vitro expansion by four donors (n=4) for 2 hours incubation.

### EMBODIMENT

Through extensive and in-depth research and a large number of process optimization experiments, the inventor has developed a method for large-scale expansion of double-negative T cells rich in Tscm and Tcm cell characteristics in vitro through serum-free medium containing composition-defined added factors. Specifically, a kind of double-negative T cells rich in Tscm and Tom cell characteristics are expanded on a large scale in vitro to further improve the allogeneic DNT cells of long-term anti-tumor effect in clinical application by providing a kind of serum-free medium derived from the peripheral blood of healthy donors, adding a composition-defined and xeno-free addition factor, and no additional soluble anti-human CD3 antibody is added in the expansion stage of the DNT culture system. On this basis, the present invention is completed.

### Terms

In order to make it easier to understand this disclosure, certain terms are first defined. As used in this application, each of the following terms shall have the meaning given below, except as otherwise expressly provided herein. Other definitions were set out throughout the application.

As used herein, the term “approximately” may refer to a value or component within an acceptable margin of error for a particular value or composition determined by a person skilled in the art, which will depend in part on how the value or composition is measured or determined.

As used herein, the terms “giving” and “administration” are used interchangeably to refer to the physical introduction of the product of the present invention into a subject using any of the various methods and delivery systems known to those skilled in the art, including intravenous, intramuscular, subcutaneous, intraperitoneal, spinal cord or other parenteral routes of administration, such as by injection or infusion.

### Amplification Method of the Present Invention In Vitro

The process flow of the present invention is as follows (FIG. 1):
(1) Isolating and purifying DNT cells from peripheral blood in vitro.
(2) Activation of DNT cells
0-2 days,
20 ng/ml-20 μg/ml of anti-human CD3 monoclonal antibody is coated in solid medium, the followings are added to the medium:
200-1000 IU/mL recombinant human interleukin 2
2%-30% (v/v) ICSR;
1-10 μg/ml of recombinant human transferrin,
1-10 μg/ml recombinant human insulin,
10-100 μg/ml ascorbic acid,
1-5 μg/ml ethanolamine,
1-5 μg/ml linoleic acid,
0.1-5 μg/ml oleic acid.
(3) Expansion of Tscm/Tcm-rich DNT cells in vitro
3-14 days, medium suitable for culturing DNT cells is used, with the addition of:
200-1000 IU/mL recombinant human interleukin 2;
0%-20% (v/v) ICSR;
one or more cytokines selected from the following groups: 10-1000 IU recombinant human interleukin 2, 5-50 ng/ml recombinant human interleukin 7, 5-50 ng/ml recombinant human interleukin 12, 5-50 ng/ml recombinant human interleukin 15, 5-50 ng/ml recombinant human interleukin 21, 1-10 ng/ml recombinant human interleukin 1β;
one or more addition factors selected from the following groups: 1-10 μg/ml of recombinant human transferrin, 1-10 g/ml of recombinant human insulin, 10-100 μg/ml of ascorbic acid, 1-5 μg/ml of ethanolamine, 1-5 μg/ml of linoleic acid, 1-5 μg/ml of oleic acid.
(4) Washing and collecting cells
14-17 days, 250 ml conical bottom centrifuge bottle is used to collect DNT cells, 900 g 10 min centrifugation, and then the cells are washed with solvent;
(5) Preparation of Tscm/Tcm-rich DNT cell preparations

The concentration is adjusted to 0.5˜1nce⁸ cells/mL with the solvent, which is the finished DNT cell preparation, which can be used for clinical use after passing the quality inspection.

In particular, in the activation of DNT cells, anti-human CD3 monoclonal antibody is coated on solid media, including but not limited to culture media, magnetic beads. Preferably, the anti-human CD3 monoclonal antibody can be coated on solid media with other antibody (e.g. anti-human CD3 monoclonal antibody) to active DNT cells. In a specific embodiment of the present invention, a CD3/CD28 magnetic bead is used to activate DNT cell, wherein the CD3/CD28 magnetic bead comprises a monoclonal antibody coated with an anti-human CD3 monoclonal antibody and an anti-human CD28 monoclonal antibody on the surface of the same magnetic bead, or two kinds of magnetic beads are coated with an anti-human CD3 monoclonal antibody and an anti-human CD28 monoclonal antibody, respectively.

Tscm/Tcm-rich DNT cells are expanded in vitro without anti-human CD3 antibodies and, preferably, soluble anti-human CD3 monoclonal antibodies, such as OKT3, are not added.

### DNT Cell Population

The present invention provides a double negative T cell (DNT) enriched with Tscm (Stem Cell-Like Memory T) and Tcm (Central Memory T, central memory T) characteristics, i.e., a DNT cell population of the present invention. Wherein, the DNT cell population is prepared by the method described in the first aspect of the present invention. It has one or more characteristics selected from the following group:
(a1) 40%-80% of cells were Tscm cells;
(b1) 10%-40% of cells are Tcm cells; or
(a2) 45%-75% of cells were Tscm cells;
(b2) 5% to 35% of cells are Tcm cells; or
(a3) 30%-60% of cells were Tscm cells;
(b3) 15%-40% of cells are Tcm cells; or
(a4) 20%-40% of cells were Tscm cells;
(b4) 20%-30% of cells are Tcm cells.

In another preferred embodiment, the proportion of Tscm cells and Tom cells of the DNT cell population [symbol] the DNT cell population more preferably 1 populatiomost preferably 1 popu

In another preferred embodiment, the DNT cell viability rate t cells;resent invention.more preferably ell viabil most preferably ll via

In another preferred embodiment, the purity of the DNT cells (CD3+) were Is (preferred embodiment more preferably preferred most preferably referre

In another preferred embodiment, the purity (%) of the DNT cells (CD3⁺CD4⁻CD8⁻) was e purity (%) of the DNT more preferably (%) of th most preferably [symbol] 97%.

In the DNT cell population of the present invention, the ratio of Tscm and Tem cells is significantly or extremely significantly better than that of cells in the prior art, and the proportion of Teff effector killer cells is significantly or extremely significantly lower than that of the prior art. Therefore, it has a stronger ability to renew, differentiate and survive.

### Pharmaceutical Composition and Methods of Administration

The invention also provides a use for a DNT cell containing the present invention for the preparation of a pharmaceutical composition or preparation that may be used to treat a disease selected from the following groups: tumor, infectious disease, autoimmune disease, graft-versus-host disease.

In another preferred embodiment, the hematologic malignancies were selected from the following groups: lymphoma (Hodgkins and non-Hodgkins), acute myeloid leukemia (AML), multiple myeloma (MM), chronic lymphocytic leukemia (CLL), acute lymphoid leukemia (ALL), diffuse large B-cell lymphoma (DLBCL), chronic myeloid leukemia (CML), chronic myelomonocytic leukemia (CMML), myelodysplastic syndrome (MDS), and a combination thereof.

In another preferred embodiment, the solid tumors were selected from the following groups: gastric cancer, gastric cancer peritoneal metastasis, liver cancer, leukemia, kidney tumors, lung cancer, small bowel cancer, melanoma, bone cancer, prostate cancer, colorectal cancer, breast cancer, colorectal cancer, colorectal cancer, cervical cancer, ovarian cancer, lymphoma, nasopharyngeal cancer, adrenal tumors, bladder tumors, non-small cell lung cancer (NSCLC), glioma, head and neck cancer, pancreatic cancer, and a combination thereof.

In another preferred embodiment, the autoimmune diseases include diabetes mellitus, rheumatoid arthritis, multiple sclerosis, systemic lupus erythematosus, nausea anemia, hemolytic anemia, autoimmune thrombocytopenia, autoimmune liver disease, ankylosing spondylitis, myasthenia gravis, IgA nephropathy, primary renal ice syndrome, psoriasis, vitiligo

The pharmaceutical composition of the present invention contains a safe and effective amount of the fusion protein of the present invention and a pharmaceutically acceptable carrier. Such carriers include, but are not limited to, saline, buffer solution, glucose, water, glycerin, ethanol, powder, DMSO or the combination thereof. In general, the pharmaceutical preparation should be matched with the administration mode, and the pharmaceutical composition of the present invention can be prepared in the form of injection, for example, prepared by a conventional method using physiological saline or an aqueous solution containing glucose and other adjuvants. The pharmaceutical composition is preferably manufactured under sterile conditions. The dosage of the active ingredient is a therapeutically effective amount. The pharmaceutical formulation of the present invention may also be made into a sustained release formulation.

For the pharmaceutical composition of the present invention, the desired object (such as human and non-human mammals) may be administered in a conventional manner. Typical administration methods include (but are not limited to): intravenous injection, arterial injection, thoracic cavity, abdominopelvic cavity, subarachnoid space, sinus, intracranial, different tissues (such as tumor tissue, inflammatory lesion tissue) and other different parts of the injection.

The main advantages of the present invention include:

(1) The expansion process is simple and stable, and it can realize large-scale culture of DNT cells rich in Tscm and Tcm cell characteristics in vitro, and the purity of DNT cells in the final product is (CD3 CD4 CD8″)≥85%, which meets the needs of clinical use.

(2) The addition components are defined, and the use of serum-free medium with defined composition in the whole culture process, and the addition of animal or human origin and other unclear components of serum or plasma are not added in the culture process, providing a better guarantee for the clinical application safety of allogeneic DNT cells, making it a truly quality-controlled, safe and reliable universal cell therapy product.

(3) DNT cells rich in Tscm and Tcm cell characteristics were cultured, and the proportion of Tscm cells and Tcm cells of DNT cells is ≥50%.

(4) There is no need to add murine anti-human CD3 antibody (clone OKT3) in the amplification stage of the DNT culture system, which further reduces the xenoprotein residue in the final product and improves the safety and quality of the product.

(5) Cells can be expanded in vitro from freshly enriched DNT cells or revived DNT cells after cryopreservation.

The present invention is further stated below in conjunction with specific embodiments. It should be understood that these embodiments are intended only to illustrate the invention and not to limit the scope of the invention. Experimental methods for which detailed conditions are not indicated in the following embodiments, usually according to the conditions described in conventional conditions such as Sambrook et al., Molecular Cloning: Laboratory Handbook (New York: Cold Spring Harbor Laboratory Press, 1989), or according to the conditions recommended by the manufacturer. Percentages and servings are by weight unless otherwise stated.

### Example 1: Anti-Human CD3 Monoclonal Antibody Activates DNT Cells Expansion Culture In Vitro

### 1.1 DNT Cell Enrichment of Healthy Donors

20˜200 ml of peripheral blood was collected from healthy donors into a tube containing sodium heparin. Using the Rossettsep® kit (Stem Cell Technologies Inc.), CD4⁺ and CD8+ T cells were removed, to obtain the cells that CD4+ and CD8+ depleted DNT cells.

### 1.2 Activation of DNT Cells

On days 0-2, the T25 flasks were coated with anti-human CD3 monoclonal antibody (10 μg/mL) (clone number OKT3), and the DNT cells obtained in step 1.1 were added with 10% (v/v) ICSR and 500 IU/mL of recombinant human interleukin 2 by DNT-cell-specific serum-free medium (AIM-V basal medium, add 8 μg/ml of recombinant human transferrin, 8 μg/ml of recombinant human insulin, 30 μg/ml of ascorbic acid, 1.5 μg/ml of ethanolamine, 1 g/ml of linoleic acid, 1 μg/ml of oleic acid) to be adjusted a concentration of 1×10⁶˜4×10⁶ cells/mL, and placed in the aforementioned coated T25 culture flask. The cells were incubated in a 5% CO₂ incubator at 37° C.

### 1.3 Expansion of DNT Cells Rich in Tscm and Tcm Cell Characteristics In Vitro

On days 3-6, 10% (v/v) ICSR and 500 IU/mL of recombinant human interleukin 2, 2 ng/ml recombinant human interleukin 18, 5 ng/ml recombinant human interleukin 21 were added to serum-free medium for DNT cells (AIM-V basal medium with 8 μg/ml recombinant human transferrin, 8 g/ml recombinant human insulin, 30 μg/ml ascorbic acid, 1.5 μg/ml ethanolamine, 1 g/ml linoleic acid, 1 g/ml oleic acid) to adjust the DNT cells to a concentration of 1×10⁶˜4×10⁶ cells/mL, and the cells were adjust to T75 flasks or T175 flasks according to the cell volume to continue culture.

On days 7-14, 5% (v/v) ICSR and 500 IU/mL of recombinant human interleukin 2, 5 ng/ml recombinant human interleukin 7.5 ng/mL ml of recombinant human interleukin 12 were added by DNT cell serum-free medium (AIM-V basal medium were added 5 ng/ml Recombinant Human Transferrin, 5 μg/ml Recombinant human insulin, 50 μg/ml ascorbic acid, 1.5 μg/ml ethanolamine, 1 g/ml linoleic acid, 1 μg/ml oleic acid) to adjust the DNT cells to a concentration of 1×10⁶˜4×10⁶ cells/mL, and then adjusting the T175 culture flask or 2 L culture bag according to the cell volume to continue culture.

On the days 13˜14, DNT cells were harvested as needed.

### 1.4 Harvesting DNT Cells to Make DNT Cell Preparations

On the 13˜14 days, DNT cells were harvested as needed. Cells were collected in a 250 ml curonged centrifuge flask, were centrifuged at 900×g for 10 min, and then washed with solvent. The collected DNT cells were adjusted to a concentration of 0.1˜1×10⁸ cells/mL with solvent as the finished DNT cell preparation, which can be used for clinical use after passing the quality inspection.

The experimental results are shown in FIG. 2-FIG. 6, Table 1, wherein,

(1) The growth curves of DNT cells (FIG. 2A) and cell viability (FIG. 2B) were detected by AO/PI staining on days 7, 9, 11, and 14 of expansion.

The results showed that on days 7, 9, 11, and 14, the expansion folds of cells in vitro were as high as 74 times, 416.6 times, 2233.8 times and 8071.5 times, respectively, and the survival rates of cells were 91.93%, 95.43%, 87.99% and 87.55%, respectively.

(2) Flow cytometry was used to detect the purity curves of DNT cells on days 7, 9, 11 and 14, as well as the Flow Cytometry Graph of CD3+% and CD3 CD4 CD8% of DNT cells on day 11. The results are shown in FIG. 3, where FIGS. 3A and 3B show the DNT cell purity curves (CD3+%, CD3⁺CD4⁻CD8⁻%) and FIG. 3C shows the DNT cell purity flow cytometry on day 11.

As shown in the table below, the results show that using the culture method of the present invention, high DNT cell purity (CD3+%=97.80%, CD3⁺CD4⁻CD8⁻%=93.) was obtained on the day 11 in vitro.

(3) Using fluorescein-labeled anti-human CD45RA/CD62L antibody, the ratio of Tscm/Tcm/Tcm/Teff cells on days 7, 9, 11 and 14 of DNT cell expansion was detected by flow cytometry (FIG. 4A) and Tscm/Tcm/Flow cytometry of Tem/Teff cell differentiation on day 11 (FIG. 4B).

The results show that the proportion of DNT cell Tscm obtained on day 11 in vitro expansion was 48.10%, and the proportion of Tcm is 29.60%.

TABLE 1
DNT cell expansion fold, viability, purity,
and DNT cell differentiation ratio

| | Day 0 | Day 7 | Day 9 | Day 11 | Day 14 |
|---|---|---|---|---|---|
| Folds of cell | 1 | 74 | 416.6 | 2233.8 | 8071.5 |
| expansion in vitro | | | | | |
| Cell viability % | 92.40 | 91.93 | 95.43 | 87.99 | 87.55 |
| DNT cells (CD3+) | 16.5% | 94.70 | 95.40 | 97.80 | 97.40 |
| purity %. | | | | | |
| DNT cells | 90.3% | 87.90 | 91.90 | 93.70 | 93.20 |
| (CD3⁺CD4⁻CD8⁻) | | | | | |
| purity % | | | | | |
| Tscm % | / | 69.90 | 48.40 | 48.10 | 57.80 |
| Tcm % | / | 8.20 | 32.40 | 29.60 | 12.60 |
| Tem % | / | 6.90 | 10.80 | 12.10 | 8.50 |
| Teff % | / | 15.00 | 8.40 | 10.20 | 21.60 |

(4) After labeling MV411 cell lines with PKH-26, they were co-incubated with DNT cells on the 10th day of in vitro expansion at 4:1 ratio for 2 hours. Flow cytometry was used to gate label MV411 cells and analyze target cell apoptosis to evaluate the tumor killing activity of DNT cells against MV411 cells (FIG. 5).

The results show that DNT cells (on the 10th day of in vitro expansion) were incubated with MV411 for 2 hours at a ratio of 4:1 for 2 hours, and the tumoricidal activity against MV411 cells is as high as 92.3%.

(5) The RTCA method is used (integrate the microelectronic cell sensor chip into the bottom of the cell detection plate through a special process to build a cell impedance detection sensing system that tracks changes in cell morphology and proliferation and differentiation in real time, dynamically, and quantitatively) to detect the real-time tumoricidal curve of DNT cells against Hela cells (FIG. 6).

The results showed, DNT cells (on the 10th day of in vitro expansion) were co-incubated with HeLa cell at efficiency target ratio of 5:1 overnight, and the cell tumoricidal activity was 69.1% on the 20th hour of co-incubation.

### Example 2: Magnetic Bead-Activated DNT Cell Expansion Culture In Vitro

### 2.1 DNT Cell Enrichment of Healthy Donors

See section 1.1 in Example 1

### 2.2 Activation of DNT Cells

On days 0-2, the isolated and purified DNT cells (beads: DNT cells=1:1) were mixed with CD3/CD28 magnetic beads (monoclonal antibody against human CD3 and monoclonal antibody against human CD28 on the surface of the same bead, or monoclonal antibody against human CD3 and monoclonal antibody against human CD28, respectively) and then inoculated in T25 flasks, and 20% (v/v) ICSR and 1000 IU/mL of recombinant human interleukin 2 were added by DNT cell serum-free medium (GT551 basal medium was added 3 μg/ml recombinant human transferrin, 3 μg/ml recombinant human insulin, 70 μg/ml ascorbic acid, 2 μg/ml ethanolamine, 0.5 g/ml linoleic acid, 0.5 g/ml oleic acid) to be adjusted with a concentration to 1×10⁶˜4×10⁶ cells/mL and put it into the above-coated T25 flask. The cells were incubated in a 5% CO₂ incubator at 37° C.

### 2.3 Expansion of DNT Cells Rich in Tscm and Tcm Cell Characteristics In Vitro

On days 3-6, 20% ICSR and 1000 IU/mL of recombinant human interleukin 2, 5 ng/ml recombinant human interleukin 1β, 3 ng/ml recombinant human interleukin 21 were added to serum-free medium for DNT cells (GT551 basal medium with 3 μg/ml recombinant human transferrin, 3 μg/ml recombinant human insulin, 70 g/ml ascorbic acid, 2 μg/ml ethanolamine, 0.5 μg/ml linoleic acid, 0.5 μg/ml oleic acid) to adjust the concentration of 1×10⁶˜4×10⁶ cells/mL, and the cells were adjust to T75 flasks or T175 flasks according to the cell volume to continue culture.

On days 7-14, 1000 IU/mL of recombinant human interleukin 2, 5 ng/ml recombinant human interleukin 7, 5 ng/ml recombinant human interleukin 15 were added to serum-free medium for DNT cells (GT551 basal medium with 3 μg/ml recombinant human transferrin, 3 μg/ml recombinant human insulin, 70 μg/ml ascorbic acid, 2 μg/ml ethanolamine, 0.5 μg/ml linoleic acid, 0.5 μg/ml oleic acid) to adjust the concentration of DNT cells to 1×10^{6˜4×10}° cells/mL, and adjusting the T175 flask or 2 L culture bag according to the cell volume to continue to culture.

On days 13˜14, DNT cells were harvested as needed.

### 2.4 Harvesting DNT Cells to Make DNT Cell Preparations

On the 13˜ 14 days, DNT cells were harvested as needed. Cells were collected in a 250 ml curonged centrifuge flask, were centrifuged at 900×g for 10 min, and then washed with solvent. The collected DNT cells were adjusted to concentration of 0.5˜1×10⁸ cells/mL with a solvent as the finished DNT cell preparation, which could be used for clinical use after passing the quality inspection.

The experimental results are shown in FIGS. 7-11, wherein,

(1) The growth curve of DNT cells (FIG. 7A) and cell viability (FIG. 7B) were detected by AO/PI staining on days 7, 9, 11 and 14 of amplification.

(2) CD3/CD4/CD8 antibody labeling, flow cytometry was used to detect the purity curve of DNT cells on day 7, day 9, day 11 and day 14, and the CD3% and CD3 CD4 CD8% of DNT cells on day 11 Flow Cytometry Graph. FIGS. 8A and 8B show the DNT cell purity change curve, and FIG. 8C shows the DNT cell purity flow cytometry plot on day 11. As shown in the table below, the results show that using the culture method of the present invention, the purity of DNT cells obtained on the 11th day of in vitro expansion is CD3+%=98.50%, CD3*CD4 CD8-%-89.20%.

(3) Fluorescein-labeled anti-human CD45RA/CD62L antibody was detected by flow cytometry to detect the ratio of Tscm/Tcm/Tem/Teff cells on day 7, day 9, day 11, and day 14 of DNT cell expansion (FIG. 9A) and day 11 of Tscm/Tcm/Tem/TeffFlow cytometry of cell differentiation (FIG. 9B).

The experimental results are shown in Table 2 below:

TABLE 2
DNT cell expansion fold, viability, purity,
and DNT cell differentiation ratio

| | Day 0 | Day 7 | Day 9 | Day 11 | Day 14 |
|---|---|---|---|---|---|
| Folds of cell | 1 | 79.65 | 734.91 | 4377.92 | 16116.3 |
| expansion in vitro | | | | | |
| Cell viability % | 92.40 | 90.16 | 95.77 | 94.37 | 93.44 |
| DNT cells (CD3+) | 16.5 | 96.40 | 95.90 | 98.50 | 98.10 |
| purity %. | | | | | |
| DNT cells | 90.3 | 72.50 | 82.50 | 89.20 | 90.90 |
| (CD3⁺CD4⁻CD8⁻) | | | | | |
| purity % | | | | | |
| Tscm % | / | 73.70 | 57.00 | 46.30 | 58.20 |
| Tcm % | / | 9.30 | 25.60 | 29.00 | 13.40 |
| Tem % | / | 4.50 | 8.10 | 14.00 | 9.50 |
| Teff % | / | 12.50 | 9.30 | 10.80 | 18.90 |

(4) After PKH-26 labeling, the MV411 cell line was incubated with a 4:1 ratio of DNT cells (on the 10th day of in vitro expansion) for 2 hours, and MV411 was gated by flow cytometry to analyze the apoptosis of the target cells and the tumor killing activity of DNT cells against MV411 cells (FIG. 10).

The results showed that the tumoricidal activity of DNT cells (on the 10th day of in vitro expansion) against MV411 cells was 34.80% when the ratio was 4:1 and the total incubation was 2 hours.

(5) The RTCA method was used to detect the real-time tumor killing curve of DNT cells against Hela cells (FIG. 11).

The results showed that DNT cells (on the 10th day of in vitro expansion) were co-incubated with Hela cell at efficiency target ratio of 5:1 overnight, and the tumoricidal performance of DNT cells was 65.40% on the 20th hour of co-incubation.

### Example 3: Anti-Human CD3 Monoclonal Antibody Activates DNT Cells In Vitro Expansion Culture

### 3.1 DNT Cell Enrichment of Healthy Donors

See section 1.1 in Examplel

### 3.2 Activation of DNT Cells

The T25 flasks were coated with anti-human CD3 monoclonal antibody (10 μg/mL), and the DNT cells obtained in step 1.1 was added with 10% ICSR and 500 IU/mL of recombinant human interleukin 2 with serum-free medium for DNT cells (Aly505 basal medium was added with 5 μg/ml of recombinant human transferrin, 5 μg/ml of recombinant human insulin, 50 μg/ml of ascorbic acid, 1.5 μg/ml of ethanolamine, 1 g/ml of linoleic acid, 1 μg/ml of oleic acid) to be adjusted to concentration of 1×10⁶˜4×10⁶ cells/mL, and the cells were placed into the above-mentioned coated T25 culture flask. The cells were incubated in a 5% CO₂ incubator at 37° C.

### 3.3 In Vitro Expansion of DNT Cells Rich in Tscm and Tcm Cell Characteristics

On days 3-6, 10% ICSR and 500 IU/mL of recombinant human interleukin 2, 5 ng/ml recombinant human interleukin 7, 5 ng/ml recombinant human interleukin 15 were added by DNT cell-specific serum-free medium (Aly505 basal medium with 5 μg/ml recombinant human transferrin, 5 μg/ml recombinant human insulin, 50 g/ml ascorbic acid, 1.5 μg/ml ethanolamine, 1 μg/ml linoleic acid, 1 μg/ml oleic acid) to adjust the DNT cells to a concentration of 1×10⁶˜4×10⁶ cells/mL, and the cells were adjust to T75 flasks or T175 flasks according to the cell volume to continue culture.

On days 7-14, 10% ICSR and 500 IU/mL of recombinant human interleukin 2, 5 ng/ml recombinant human interleukin 7, 5 ng/ml recombinant human interleukin 15 were added by DNT cells-specific serum-free medium (Aly505 basal medium with 3 μg/ml recombinant human transferrin, 3 μg/ml recombinant human insulin, 30 g/ml ascorbic acid, 1 μg/ml ethanolamine, 0.5 μg/ml linoleic acid, 0.5 μg/ml oleic acid) to adjust the DNT cells to 1×10⁶˜4×10⁶ cells/mL concentration, and adjust the T175 culture flask or 2 L culture bag according to the cell volume to continue culture.

On days 13˜14, DNT cells were harvested as needed.

### 3.4 Harvest DNT Cells to Make DNT Cell Preparations

On days 13˜14, DNT cells were harvested as needed. The cells was collected in a 250 ml curonged centrifuge flask, centrifuged at 900×g for 10 min, and then washed with solvent. The collected DNT cells were adjusted to a concentration of 0.5˜1×10⁸ cells/mL with a solvent, which was a finished DNT cell preparation, which could be used for clinical use after passing the quality inspection.

The experimental results are shown in FIGS. 12-16. Wherein,

(1) The growth curve of DNT cells (FIG. 12A) and cell viability (FIG. 12B) were detected by AO/PI staining on days 7, 9, 11 and 14 of expansion.

(2) Fluorescein-labeled anti-human CD3/CD4/CD8 antibody was detected by flow cytometry to detect the purity curves of DNT cells on day 7, day 9, day 11 and day 14 of DNT cell expansion, and the CD3% and CD3 CD4 CD8% of DNT cells on day 11 of flow cytometry. FIGS. 13A and 13B show the DNT cell purity change curve, and FIG. 13C shows the DNT cell purity flow cytometry of day 11.

(3) The fluorescein-labeled anti-human CD45RA/CD62L antibody, flow cytometry was used to detect the proportion of Tscm/Tem/Tem/Teff cells on day 7, day 9, day 11, and day 14 of DNT cell expansion (FIG. 14A) and flow cytometry of Tscm/Tcm/Tem/Teff cell differentiation of day 11 (FIG. 14B).

The experimental results are shown in Table 3 below:

TABLE 3
DNT cell expansion fold, viability, purity,
and DNT cell differentiation ratio

| | Day 0 | Day 7 | Day 9 | Day 11 | Day 14 |
|---|---|---|---|---|---|
| Folds of cell | 1 | 106.38 | 824.22 | 5286.21 | 21501.42 |
| expansion in vitro | | | | | |
| Cell viability % | 92.40 | 94.11 | 91.58 | 89.13 | 89.29 |
| DNT cells (CD3+) | 16.5 | 95.60 | 95.30 | 98.80 | 99.50 |
| purity %. | | | | | |
| DNT cells | 90.3 | 86.80 | 86.60 | 86.50 | 87.00 |
| (CD3⁺CD4⁻CD8⁻) | | | | | |
| purity % | | | | | |
| Tscm % | / | 59.30 | 44.10 | 31.50 | 43.70 |
| Tcm % | / | 21.20 | 22.00 | 39.00 | 16.00 |
| Tem % | / | 7.40 | 15.20 | 21.40 | 18.00 |
| Teff % | / | 12.10 | 18.60 | 8.10 | 22.30 |

(4) PKH-26 labeled the MV411 cell line and incubated with a 4:1 ratio of DNT cells (on the 10th day of in vitro expansion) for 2 hours, and MV411 was gated by flow cytometry to analyze the apoptosis of the target cells and the tumoricidal activity of DNT cells against MV411 cells (FIG. 15).

The results showed that the tumoricidal activity of DNT cells (on the 10th day of in vitro expansion) against MV411 cells was 78.40% when the ratio was 4:1 and the total incubation was 2 hours.

(5) The real-time tumoricidal curve of DNT cells against Hela cells was detected by RTCA method (FIG. 16).

The results showed that DNT cells (on the 10th day of in vitro expansion) were co-incubated with HeLa cells with a 5:1 ratio overnight, and the tumoricidal activity was 54.30% on the 20th hour of co-incubation.

### 4. Comparison Example

The invention patent is a method for large-scale expansion of double negative T (DNT) cells enriched with Tscm (Stem Cell-like Memory T) and Tcm (Centr al Memory T) and Tom (Central Memory T, central memory T) in vitro (hereinafter referred to as the new process) is compared with our company's existing patent (CN 104109653 A), that is, the method of large-scale expansion of human peripheral blood DNT cells using an animal-free serum culture system (hereinafter referred to as the old process).

20˜200 ml of peripheral blood was collected from 4 healthy donors into a tube containing sodium heparin. The Rossettsep® kit (Stem Cell Technologies Inc.) was used to remove CD4⁺ and CD8+ T cells, and the resulting cells were CD4+ and CD8+ depleted DNT cells.

### 4.1 New Process Method to Activate and Expand DNT Cells: Example 3 Method is Used to Produce Expanded DNT Cells

### 4.2 Activation and Expansion of DNT Cells by the Old Process Method:

### 4.2.1 DNT Cell Activation In Vitro

The T25 flask was coated with anti-human CD3 monoclonal antibody (10 μg/mL), and the CD4+ and CD8+ depleted DNT cells were added to the T25 flask with DNT serum-free medium (AIM-V basal medium with 500 IU/mL of recombinant human interleukin 2, 2 ng/ml recombinant human interleukin 4, 8 ng/ml recombinant human interleukin 7, 5 ng/ml recombinant human interleukin 12.5 v % autologous serum and 6 v % autologous serum) adjusted to a concentration of 1×10⁶˜4×10⁶ cells/mL and the cells were incubated in a 5% CO₂ incubator at 37° C. for 3 days.

### 4.2.2 Expanding DNT Cells In Vitro

On days 3-6, DNT serum-free medium (AIM-V basal medium was added 500 IU/mL of recombinant human interleukin 2, 2 ng/ml recombinant human interleukin 4, 8 ng/ml recombinant human interleukin 7, 5 ng/ml recombinant human interleukin 12, 5 ng/ml autologous serum and 6 v %) was used to adjusted to a concentration of 1×10⁶˜4×10⁶ cells/mL, and adjusted to T75 flasks or T175 flasks according to the cell volume to continue culture.

On day 7, DNT serum-free medium (IM-V basal medium was added 500 IU/mL of recombinant human interleukin 2, 2 ng/ml recombinant human interleukin 4, 8 ng/ml recombinant human interleukin 7, 5 ng/ml recombinant human interleukin 12.5 v % with. autologous serum and 6 v % autologous serum) were additionally added with 50 ng/ml soluble anti-human CD3 monoclonal antibody, and adjusted to a concentration of 1×10⁶˜4×10⁶ cells/mL, and the T175 flask was adjusted according to the cell volume to continue culture.

On days 10-14, DNT serum-free medium (GT551 basal medium was added 500 IU/mL of recombinant human interleukin 2, 2 ng/ml recombinant human interleukin 4, 8 ng/ml recombinant human interleukin 7, 5 ng/ml recombinant human interleukin 12.5 v % with. autologous serum and 6 v % autologous serum) was additionally added with 50 ng/ml soluble anti-human CD3 monoclonal antibody, adjusted to a concentration of 1×10⁶˜4×10⁶ cells/mL, and T175 flasks or 2 L bags were adjusted according to the cell volume to continue culture.

On days 13˜14, DNT cells were harvested as needed.

### 4.2.3 Harvesting DNT Cells to Make DNT Cell Preparations

On the 13th˜14th day, DNT cells were harvested as needed. The cells were collected in a 250 ml curonged centrifuge flask, centrifuged at 900×g for 10 min, and then washed with solvent. The collected DNT cells were adjusted to a concentration of 0.5˜1×10⁸ cells/mL with a solvent, which was a finished DNT cell preparation, which could be used for clinical use after passing the quality inspection.

### 4.2.4 Functional Detection and Analysis of DNT Cells Amplified by New and Old Processes:

(1) AO/PI staining was used to detect the growth curves and cell viability curves of DNT cells on the 7th, 10th, and 14th days of expansion of the four donors between the new and the old process (n=4) (FIGS. 17A-B).

Compared with the old process, there was no significant difference in the expansion fold and cell viability of DNT cells on the 14th day of culture prepared by the new process.

(2) Anti-human CD3/CD4/CD8 antibody was labeled with fluorescein, and flow cytometry was used to detect the purity of DNT cells (CD3 CD4 CD8%) (n=4) on day 7, day 10, and day 14 of the new process and the old process expansion of four donors (FIG. 18).

The results are shown in FIG. 18, where FIG. 18A-B shows CD3/CD4/CD8 antibody labeling, DNT cell purity curves and differential analysis (n=4) on day 7, day 10, and day 14 of DNT cell expansion between the new process and the old process for 4 donors was detected by flow cytometry, and Flow cytometry of DNT cell purity on the 10th day of amplification using donor 2's new process (18C) and old process (18D) (CD3+% and CD3⁺CD4⁻CD8⁻%).

There is no significant difference in the purity (CD3⁺CD4⁻CD8⁻%) of DNT cells harvested on day 14 of culture using the new process compared with the old process.

(3) Anti-human CD45RA/CD62L antibody was labeled with fluorescein, and flow cytometry was used to detected the cell differentiation ratio (n=4) of Tscm/Tcm/Tem/Teff (Tscm=CD45RA⁺/CD62L⁺, Tcm=CD45RA⁻/CD62L⁺, Tem=CD45RA⁻/CD62L⁻, Tem=CD45RA⁺/CD62L⁻) that amplified on day 7, day 10 and day 14 between the new process and the old process of 4 donors, ⁻) (FIG. 19).

The results are shown in FIGS. 19 and Table 4, wherein A-C in FIG. 19 shows CD45RA/CD62L antibody labeling, and flow cytometry was used to detect the changes in the proportion of Tscm/Tcm/Tem/Teff cells and the difference analysis (n=4) of DNT cells on days 7, 10, and 14 of the new process and the old process. D-E in FIG. 19 shows the flow cytometry of DNT cell differentiation Tscm/Tcm/Tem/Teff cells from donor 2 on day 10 of expansion in vitro.

Compared with the old process, the ratio of Tscm to Tcm cells in DNT cells expanded by the new process was significantly or extremely significantly higher than that of the old process, while the proportion of Teff effector killer cells was significantly or very significantly lower than that of the old process (as shown in FIG. 19, Table 4).

TABLE 4
Proportions of Tscm, Tem, Tem and Teff
effector killer cells in DNT cells

| | | Day 7 | Day 10 | Day 14 |
|---|---|---|---|---|
| Tscm % | New process | 27.27 | 25.95 | 29.98 |
| | Old process | 6.63 | 9.60 | 10.93 |
| Tcm % | New process | 19.23 | 15.15 | 13.80 |
| | Old process | 5.80 | 7.23 | 3.28 |
| Tem % | New process | 33.87 | 29.90 | 23.10 |
| | Old process | 53.10 | 35.65 | 19.53 |
| Teff % | New process | 19.63 | 28.98 | 33.15 |
| | Old process | 31.75 | 47.23 | 65.88 |

(4) Flow cytometry was used to detect the cell tumoricidal activity of DNT cells with 4:1 ratio of DNT cells to MV411 cells on day 10 (n=4) by flow cytometry (FIG. 20).

FIG. 20 shows the cell tumoricidal activity and differential analysis of DNT cells (on day 10 of in vitro expansion by four donors) incubated with MV411 at efficiency target ratio of 4:1 for 2 hours (n=4).

The results of cell tumoricidal activity on day 10 showed that the tumoricidal activity of DNT cells obtained by the new process was significantly higher than that of the old process.

To sum up, the main points of the new process is better than the old process are:
1. Cytokines and culture components that are more suitable for the growth of DNT cells are added;
2. Murine soluble anti-human CD3 antibody was not used in the amplification stage to reduce the residue of murine components and further improve the safety of the product
3. Healthy donor plasma is replaced with a commercially sourced serum substitute (such as ICSR or KSR) throughout the production process to ensure batch-to-batch consistency of different products
4. DNT cells rich in Tscm and Tcm characteristics cultured have stronger self-renewal, differentiation and long-term survival ability, which can survive in the body for a long time, and play a long-term anti-tumor role.

All literatures mentioned in the present application are incorporated herein by reference, as though each one is individually incorporated by reference. In addition, it should also be understood that, after reading the above teachings of the present invention, those skilled in the art can make various changes or modifications, equivalents of which falls in the scope of claims as defined in the appended claims.

1. An expansion method for double-negative T cells enriched with Tscm cells and Tcm in vitro, comprising steps:
(a) providing an initial sample I of peripheral blood obtained from a donor;
(b) pretreating the initial sample I to obtain sample II;
(c) culturing sample II in a culture system containing a culture medium suitable for the growth of DNT cell, thereby obtaining a sample III, wherein one or more cytokines selected from the following group are added to the culture system:
5-50 ng/ml recombinant human interleukin 21, 1-10 ng/ml recombinant human interleukin 1β, 5-50 ng/ml recombinant human interleukin 7, 5-50 ng/ml recombinant human interleukin 15, 5-50 ng/ml recombinant human interleukin 12;
wherein, in step (c), anti-human CD3 antibody is not added to the culture system.

2. The method of claim 1, wherein the step (c) also comprises the following steps:
(d) in a culture system containing medium suitable for DNT cell growth, sample III is cultured to obtain the required amount of Tscm and Tem-rich DNT cells, as sample IV; wherein, one or more cytokines selected from the following group are added to the culture system:
5-50 ng/ml recombinant human interleukin 7, 5-50 ng/ml recombinant human interleukin 12 or 5-50 ng/ml recombinant human interleukin 15; and
(e) collecting sample IV from a solution suitable for DNT cell preservation.

3. The method of claim 1, wherein the pretreatment in step (b) comprises:
(b1) CD4+ and CD8+ T cells are removed from the starting sample I, resulting a starting sample I with CD4+ and CD8+ depleted;
(b2) sample II was obtained through activating starting sample I with CD4+ and CD8+ depleted by anti-human CD3 monoclonal antibody in a culture system suitable for DNT cell growth.

4. The method of claim 1, wherein in step (b), the number of cells of the CD4+ and CD8+ depleted starting sample I is NO;
in step (c), the number of DNT cells in sample III is N1;
in step (d), the number of DNT cells in sample IV is N2, wherein,
N1/N0r of preferably DNT more preferably DNT c most preferably DNT c
N2/N0ably DNT cells in samplemore preferably T cells most preferably T cells.

5. The method of claim 1, in step (c), the number of culture days is selected from 5-21 days, preferably 6-17 days, and more preferably 7-14 days.

6. The method of claim 1, in steps (b), (c) and (d), the culture system of the medium suitable for DNT cell growth contains serum substitutes selected from the following groups: ICSR (Immune Cell Serum Replacement), KSR (KnockOut Serum Replacement).

7. A DNT cell population, wherein the DNT cell population is prepared by the method of claim 1.

8. The DNT cell population of claim 7, wherein the DNT cell population has one or more characteristics selected from the following group:
(a1) 40%-80% of cells were Tscm cells;
(b1) 10%-40% of cells are Tcm cells; or
(a2) 45%-75% of cells were Tscm cells;
(b2) 5% to 35% of cells are Tom cells; or
(a3) 30%-60% of cells were Tscm cells;
(b3) 15%-40% of cells are Tcm cells; or
(a4) 20%-40% of cells were Tscm cells;
(b4) 20%-30% of cells are Tcm cells.

9. The DNT cell population of claim 7, wherein the purity (%) of DNT cells (CD3*) is s3 cells (of 1 populatio more preferably f 1 popula most preferably f 1 p; and/or
the purity (%) of the DNT cells (CD3⁺CD4⁻CD8⁺) was purity (%) of the DNT ce more preferably %) of the most preferably %) of

10. The use of DNT cells of claim 7 for the preparation of a pharmaceutical composition or preparation, wherein the pharmaceutical composition or preparation is used for:
(a) prevention and/or treatment of tumors;
(b) prevention and/or treatment of infectious diseases;
(c) prevention and/or treatment of autoimmune diseases;
(d) prevention and/or treatment of graft-versus-host disease; and/or
(e) modulation of immune responses.

11. The use of claim 10, wherein the tumor selected from the following groups: hematologic tumors, solid tumors, or a combination thereof.

12. The use of claim 10, wherein the hematologic malignancies selected from the following groups: lymphoma (Hodgkins and non-Hodgkins), acute myeloid leukemia (AML), multiple myeloma (MM), chronic lymphocytic leukemia (CLL), acute lymphoid leukemia (ALL), diffuse large B-cell lymphoma (DLBCL), chronic myeloid leukemia (CML), chronic myelomonocytic leukemia (CMML), myelodysplastic syndrome (MDS), or a combination thereof.

13. The use of claim 10, wherein the solid tumors selected from the following groups: gastric cancer, gastric cancer peritoneal metastasis, liver cancer, leukemia, kidney tumors, lung cancer, small bowel cancer, melanoma, bone cancer, prostate cancer, colorectal cancer, breast cancer, colorectal cancer, colorectal cancer, cervical cancer, ovarian cancer, lymphoma, nasopharyngeal cancer, adrenal tumors, bladder tumors, non-small cell lung cancer (NSCLC), glioma, head and neck cancer, pancreatic cancer, or a combination thereof.

14. The use of claim 11, wherein the autoimmune diseases include diabetes mellitus, rheumatoid arthritis, multiple sclerosis, systemic lupus erythematosus, nausea anemia, hemolytic anemia, autoimmune thrombocytopenia, autoimmune liver disease, ankylosing spondylitis, myasthenia gravis, IgA nephropathy, primary renal ice syndrome, psoriasis, vitiligo.

15. A cell preparation, wherein the cell preparation comprises a population of DNT cells of claim 7.

16. A medium suitable for the growth of DNT cells, wherein the medium comprises one or more cytokines selected from the following group:
5-50 ng/ml recombinant human interleukin 21, 1-10 ng/ml recombinant human interleukin 1β, 5-50 ng/ml recombinant human interleukin 7, 5-50 ng/ml recombinant human interleukin 15, 5-50 ng/ml recombinant human interleukin 12;
wherein, anti-human CD3 antibody is not added to the medium.
